(19) United States
(12) Patent Application Publication
(10) Pub. No.: US 2012/0283594 A1
(43) Pub. Date: Nov. 8, 2012

(54) METHOD FOR DETERMINING HEARING THRESHOLDS

(76) Inventors: Inger Uhlén, Danderyd (SE); Francisco Lacerda, Vailentuna (SE); Iris-Corinna Schwarz, Spanga (SE); Birgitta Sköld, Grodinge (SE)

(21) Appl. No.: 13/516,256

(22) PCT Filed: Dec. 15, 2010
(86) PCT No.: PCT/SE10/51388

§ 371 Date: Jul. 19, 2012

(30) Foreign Application Priority Data
Dec. 15, 2009 (SE) 0901567-8

(51) Int. Cl. A61B 5/12

(52) U.S. Cl. 600/559

(57) ABSTRACT

A method for registering a person's ability to hear, which method comprises the use of an eye tracker, wherein a person's hearing threshold is determined by emitting a sound with a determined frequency and sound intensity level to the test person, whereby the persons eye movements is followed by an eye tracker system, and a hit for said determined frequency and sound intensity level is registered when the eye tracker has registered an eye movement hit.

The present invention relates to a method of measuring and evaluating hearing, especially for small children where the hearing test normally used does not give an accurate result or cannot be used.

### BACKGROUND OF THE INVENTION

Early detection of infant hearing loss with neonatal hearing screening is now established in most developed countries. The early diagnosis of a hearing impairment is essential to maintain the chance of normal language acquisition. Current methodology to investigate hearing in infants entails objective methods such at otoacoustic emissions (OAE) and auditory brainstem response (ABR), which both can only determine the presence or absence of a neural response towards auditory stimuli. With click-evoked ABR the degree of hearing impairment can be estimated. However, it is not frequency specific and it requires a quiet or sleeping child. As soon as a hearing impairment is diagnosed, intervention should take place. Hearing aids can be fitted as early as two months of age. Programming of the hearing aid is then based on the ABR thresholds, until the child is old enough to give a distinct behavioural response to sound stimuli, typically at 4-6 months. Between 4 and 7 months of age, an infant begins to turn its head and eyes towards a sound source and the reactions can be observed, as a result of improved eye movement and neck muscle strength.

An identified problem with today's hearing test methodology is the low significance level of the results. Most children perform random movement during the test, some more, some less. The audiologist must try to distinguish true head turns from random movement, which seems to be a difficult task when the child is restless or fussy. Also, each audiologist has its own criteria for a head turn, for example there is no exact value of degrees of head turn angle that every audiologist have to follow and even if there was one it would be very difficult to measure it during the test.

Another method to test the hearing of a person is to use visual reinforcement audiometry (VRA). VRA is based upon the head-turn paradigm and involves that the infant builds up an association between the presence of a sound stimulus and a reward display. The infant's response in anticipation towards the reward at the noted presence of an auditory stimulus is then interpreted by the audiologist as a signal of hearing. This behavioural observation test suffers from poor reliability, lengthy test times across several sessions, heavy experimental bias, and interpretative ambiguity of the broad variety of possible infant responses. A method for measuring head turn response of VRA has for example been described in WO2009/149378.

Hearing test of small children is a challenge during the first 1-2 years and for children with other disabilities even longer. The current practice therefore costs the infant valuable months of exposure to auditory stimuli and therefore impacts development negatively. There is a definite need for a more objective tool to diagnose hearing impairment in the youngest, as worldwide more than 665,000 infants are diagnosed with a significant hearing loss every year. In Sweden more than 100,000 newborns are screened every year and about 2% need further investigation. 2-300 infants are diagnosed with a permanent hearing loss.

ABR with tone burst stimulation and ASSR (Auditory Steady State Response) are being used to give more frequency specific information. The variability of these responses is high and the test procedure still requires a sleeping child.

Hearing can be measured as thresholds of hearing. The absolute threshold of hearing (ATH) is the minimum sound level of a pure tone that an average ear with normal hearing can hear with no other sound present.

The threshold of hearing is generally reported as the RMS sound pressure of 20 μPa (micropascals)=2×10⁻⁵ Pascal (Pa), a reference level that is often used in human auditory science and is given a value of 0 dB Sound Pressure Level (SPL). It is approximately the quietest sound a young human with undamaged hearing can detect at 1,000 Hz. The threshold of hearing is frequency dependent and it has been shown that the ear's sensitivity is best at frequencies between 1 kHz and 5 kHz.

There are several different psychophysical methods which can be used for the measurement of absolute thresholds. These methods may vary in many ways; however, certain aspects are identical. Firstly, the stimulus is defined, and the manner by which the person should respond is clearly specified. The sound is then presented to the listener and the level of the stimulus is manipulated in a predetermined pattern. The absolute threshold is defined statistically, often as an average of all obtained hearing thresholds.

Procedures that has been used contains a series of trials, with each trial using the ‘single-interval “yes”/“no” paradigm’. This means that sound may be present or absent in the single interval, and the listener has to say whether he thought the stimulus was there.

It is however a problem to use these methods for persons that do not have the possibility to respond “yes” or “no”, for example young children or people with cognitive or speech disturbances.

One solution to this problem is to use equipment to follow the eye-movement of the test person. This has for example been described in WO2008/046091 where a method for screening eye movement is described as a help in the treatment of deficits in auditory processing. The method according to WO2008/046091 comprises a sound-emitting device capable of emitting sound patterns to measure a young infants risk for Language Learning Impariments (“LLI”); a processor operably connected to the sound-emitting device; an input device, operably connected to the processor and capable of initiating or modifying the pattern of auditory stimuli and initiating a reinforcement stimulus; and a recorder adapted to register a response of the infant. The system may also contain a screen and the reinforcement stimulus may be a video. To register eye-movements one can use a video camera or a digital camera. One embodiment shows an eye tracker, or eye tracking system, i.e. an automated eye gaze tracking system, including a microcomputer with one or more display screens, aural loudspeakers, an eye image camera and hardware and software components to deliver the method and recognize the infants visual point by tracking movement of their eyes. Eye movements or head movements may also be recorded by human observer or by electrophysiological measurements of muscle activity. The method according to WO2008/046091 is described to record if the test person can hear the emitted sound.

A problem with the method according to WO2008/046091 is that it only tests the ability of a person to hear, it does not determine the hearing thresholds. Hearing thresholds describe the degree of hearing impairment and the need for intervention.

U.S. Pat. No. 6,461,297 describes another eye tracking system that is adapted to be used as in a computer network environment, wherein home-based therapeutic and evaluative regimens of patients can be monitored and managed from a central client, i.e. a therapist, or other authorized clinical or medical personnel. The therapeutic apparatus, to be worn by the patient, comprises a unit to be placed on the head of the patient. The unit is equipped with features for providing audible and/or visual stimuli to the patient. The unit is also equipped with head phones and a microphone and a sensor that can monitor eye and head movements. The method in U.S. Pat. No. 6,461,297 is adapted to diagnose and evaluate a patient's dizziness or balance disorder and does not describe how eye movements is tracked or what it could be used for. A problem with this method is also that the unit to be worn by the patient is not suitable for children or infants and it is not adapted to measure hearing of emitted sounds.

Accordingly there remains a need for a method that can be used to measure hearing thresholds, especially in young children, to determine the degree of hearing impairment. Hearing thresholds for a number of key frequencies constitute the basis for assessment with a hearing device.

The present invention solves the above described problems.

Thus, the present invention relates to a method for registering a person's ability to hear, by measuring their hearing thresholds, wherein the method comprises use of an eye tracker.

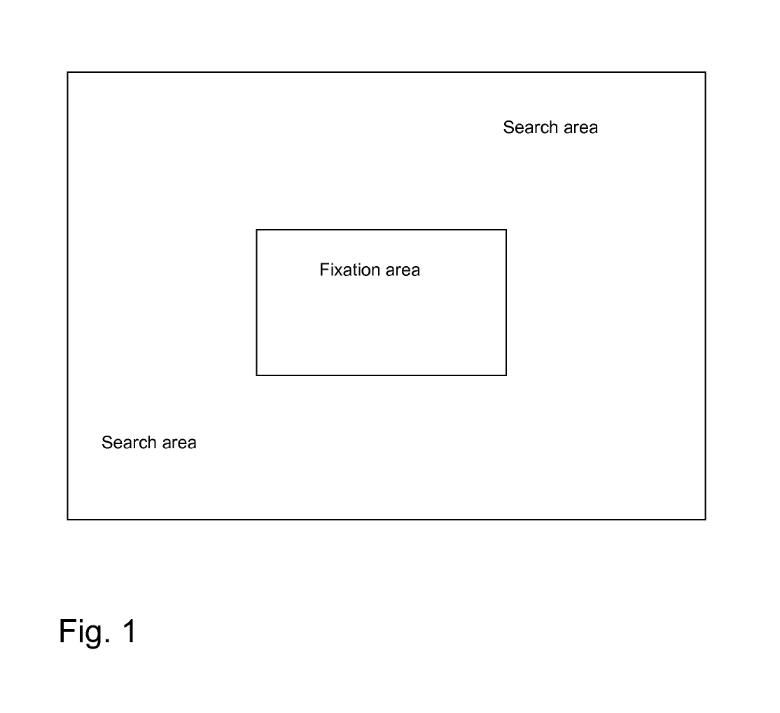

FIG. 1 shows the search area and the fixation area of the eye tracker screen.

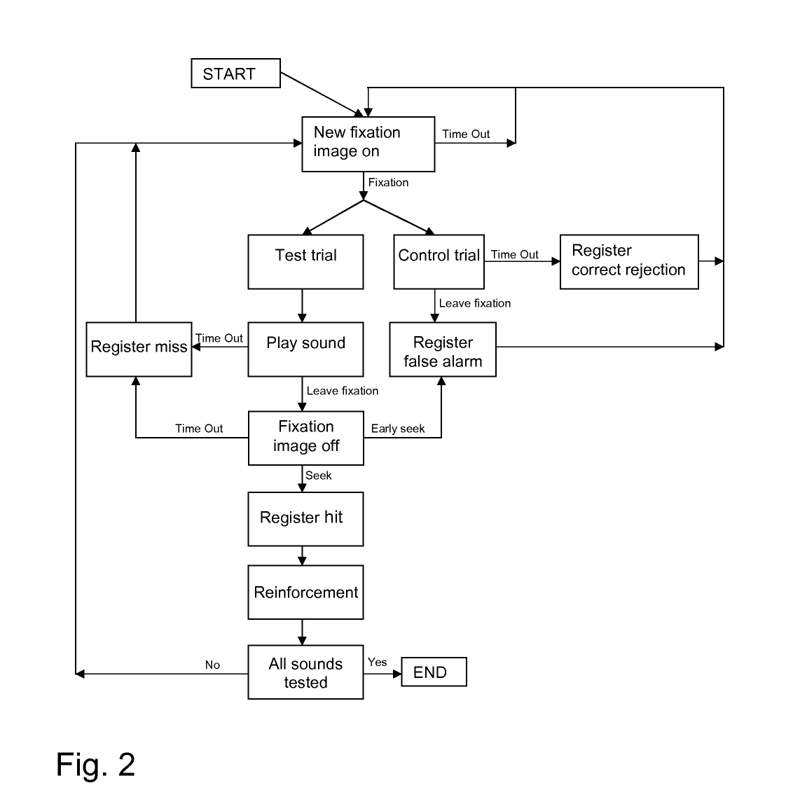

FIG. 2 shows an example of a flow chart for a test procedure.

### DETAILED DESCRIPTION OF THE INVENTION

The method according to the present invention uses an eye tracking equipment that can tell where a person is looking on a monitor by reading the gaze position, for example by using the reflection from the eye of invisible infrared light. With this technology rapid changes in the infant's gaze points can be explored as a behavioral response with shorter latency than the slow head movements traditionally used in VRA. The target, reinforcement and fixation will now all be presented on the monitor connected to the eye tracker.

In the method according to the present invention an eye tracker and a computer program is the classifier of hits and misses, instead of the test controller. The methodology is similar to VRA with the infant's response in anticipation towards a reward at the noted presence of an auditory stimulus, but instead of head turns, eye movements are registered.

Since all gaze movement data during the test is collected and saved by the eye tracker, the method according to the present invention will detect signs of hearing in a more careful way than with current methodology.

The method is also expected to improve time efficiency by performing and evaluating each trial faster. Shorter latencies can be expected to result in a shorter total testing time. This allows for higher number of observations and better significance level.

The facts that the eye tracker does not need head-mounted equipment, that it is tolerant to head movements, and even permits the user to look away from the eye tracker without needing recalibration, still with high accuracy, are compelling reasons for using it in infant investigation.

In the method according to the present invention, a test person is placed in front of a screen, for example an eye tracker screen, and a sound is emitted to the test person using insert earphones, head phones, speakers, or any other sound emitting equipment.

The test person placed in front of the eye tracker screen is presented with an interesting image in the middle of the screen, called the fixation image, which image is trying to get the test persons visual attention. The screen area surrounding the fixation image is defined as fixation area (see FIG. 1). All screen area outside the fixation area is defined as seek area (see FIG. 1).

The eye tracker delivers gaze data to a computer program which determines if the gaze is within the fixation area or in the seek area.

When the participant looks at the fixation image a sound is presented and the participant is supposed to react by moving its gaze from the fixation area to the seek area.

If the participant leaves the fixation image with its gaze the fixation image is turned off. If the participant looks within the seek area, a funny animated image is moving along the edge of the screen as reinforcement. Time criteria are set for minimum reaction time, maximum leave fixation time and seek reinforcement time.

With this methodology, the idea is that the participant will eventually anticipate the reinforcement upon hearing the sound. When the participant hears a sound, it will start looking for the reinforcement in the seek area. If it does not hear a sound, the focus should remain in the fixation area, looking at the fixation image in the middle of the screen. These two outcomes will determine whether the participant heard the presented sound or not.

The test person needs to learn the procedure of the test. Therefore, a conditioning phase is initiated before the actual test can start.

Time criteria are set to define the different steps in the test procedure. Sound is delivered when the gaze has been within the fixation area for a time duration set by a predetermined fixation time, most preferably 600 ms+randomized amount between 0 ms and 700 ms to obtain an infrequent presentation of sound stimuli. The reaction time when a gaze movement can be related to a stimulus is estimated to be about 200 ms. It is preferably set to 75% of the average reaction time the participant had during the conditioning phase. The time interval for an accepted response is from 200 ms±100 ms up to 3000 ms. The gaze then has to stay in the seek area for a defined time period before the reinforcement is displayed (edge time). The edge time is set to 300 ms but can have other values.

If the participant looks away from the screen a flashing image (attention grabber) can be launched to catch the gaze back to the screen. This screen out time is set to 3000 ms±1500 ms.

A computer program analyzes gaze data from an eye tracker, turns on and off fixation images and reinforcements on the eye tracker screen depending on the test persons gaze point, presents different sounds and produce results.

To make the hearing test more accurate, control trials and a variable of fixation time is added to the methodology. The additions results in four different outcomes of each trial; hit, miss, correct rejection or false alarm.

### Hit

A hit is registered if the participant has fixated its gaze on the fixation image, a sound has been presented and the participant has moved its gaze to seek area and fixated the gaze there in a sufficient time period of about 300 ms. Then the computer program analyzes that behavior as looking for reinforcement. The interesting animation is shown as reinforcement.

### Miss

A miss is registered if the participant does not leave the fixation image with its gaze within a time set or if the participant does not fixate its gaze in the seek area long enough.

### Correct Rejection

A control trial is a trial where no sound is presented when gaze focus is on the fixation image. The purpose is to assess potential bias towards looking to the reinforcement even in the absence of sound. The reinforcement can only be shown when a sound has been presented, never during a control trial. The “correct” reaction during a control trial is to maintain gaze fixation on the fixation image until the next trial starts.

### False Alarm

If the participant moves its gaze focus from the fixation image during a control trial a false alarm will be registered.

A false alarm can also be registered during a test trial. If a hit is registered before reasonable reaction time the outcome will instead be registered as a false alarm.

The trials are automatically collated to an objective audiogram displaying the individual hearing curve for all tested frequencies with their corresponding significance level.

### Stepping Through Sound Levels

An audiogram consists of hearing thresholds for different frequencies. To produce an audiogram, a level of sound intensity, a hearing threshold has to be determined for each frequency. This is done by stepping between different sound levels and trying to find the test persons thresholds.

A non-limiting embodiment of the invention will now be described in more detail.

A starting frequency of 1000 Hz could for example be used. A start sound of 40 dB is given to the test person.

If a miss is registered, the sound intensity level is increased by 5 dB. If a hit is registered, the sound intensity level is decreased by 10 dB. This procedure is repeated until the significance criteria for the lowest sound intensity level has been reached.

To quickly find an approximate value for the threshold, the program makes bigger steps in the beginning.

If the program starts at 40 dB HL and a miss is registered, it will step up 20 dB for each miss up to 60 dB. Over 60 dB the steps become smaller, 10 dB, and over 80 dB the steps are only 5 dB since these values of sound level are high and might hurt the test person's ears if the steps are bigger. When the first hit has been registered for the current frequency, the stepping procedure is set to standard as described above (up 5 dB for miss, down 10 dB for hit).

If the program starts at 40 dB HL and the test person registers a hit, the sound intensity is lowered to 20 dB to see if this also gives a hit. When the first miss has been registered for the current frequency, the stepping procedure is set to standard as described above (up 5 dB for miss, down 10 dB for hit).

### Stepping Through Frequencies

To decrease the significance level of the results, more than one hit for each sound level is required to determine the hearing threshold. One non-limiting example for calculating the significance level is by using the binominal probability distribution:

pₓ(k)=(ₖⁿ)p^{k}(1−p)^{n-k}, k=1, 2, . . . , n,

An assumption is made that the possibility for a hit is almost equal to the possibility for a miss, but slightly lower since the application has control trials, see the following equation:

$${p}_{hit} = 0.5 \frac{\# test trials}{\# test trials + \# control trials} = 0.45$$

The probability value for a miss is therefore 0.55 as shown in the following equation:

pₘᵢₛₛ=1−pₕᵢₜ=0.55

The test will continue to step through sound levels until the significance level for a sound level is matching three hits in a row, as the following equation shows.

pₓ(3)=(₃³)0.45³(1−0.45)³⁻³=0.091125

pₓ(k)<0.091125→Significance approved

When that criteria is met, the sound intensity level is noted as the threshold of that specific frequency. This means that the probability for a random hit, at this time, is less than 10%. If the value of approval is higher, the test results become more uncertain but the test will not require as many trials and thereby have a shorter duration. There is a balance between the desired value of significance level and the duration of the test.

This method could make the test go on forever before a threshold is set since the value of approval requires a certain amount of hits in a row or a small amount if misses (according to the binomial probability distribution described above). A miss once in a while will impair the significance level and require more hits on that specific sound intensity level. Because of this, a time limit is set for each frequency. If the time limit is exceeded, the sound intensity level with the lowest significance level is set as the threshold for that frequency. The total time limit is 900,000 ms, most preferably 300,000 ms with regard to the short attention span of a child. The total time limit is divided by the number of frequencies tested. If only one ear is tested total time is divided by two. The test continues with next frequency when a significant threshold has been determined. The significance level of the threshold for each frequency is printed in the result.

It should be evident to a person skilled in the art that any appropriate equations for determining the significance level can be used.

Infants gain control over their eye movements earlier than over their head, permitting the use of an eye tracking system at an earlier test age. As soon as the eye tracking equipment can calibrate infant eyes, their hearing can be tested. Eye movements have such fast response times even in very young infants that the total hearing test time is reduced from around 45 min (in several sessions) down to 5 min, corresponding more realistically to the short infant attention spans. Result objectivity is improved by increasing the number of test trials for each frequency and hearing level, as well as by providing a significance level for each tested frequency depending on the number of test trials. High test reliability and experimenter independence are achieved by the program's automatic detection of the infant response. Experimenter involvement is not necessary as the program automatically registers all responses and adapts the stimulus levels for each frequency according to the previous infant responses which also reduces the potential for human error.

Variations of the disclosed arrangement are possible without departing from the invention. For example, although the invention has been described using a screen to visualize the stimuli, it will be appreciated that the fixation image and seek area could also be embodied by other means. Accordingly the above description of the specific embodiment is made by way of example only and not for the purposes of limitation.

Although the disclosure has been mostly described for infants, it is also obvious for person skilled in the art that the described method can be used for any person to determine the persons hearing thresholds.

Thus, the invention shall not be limited to the described embodiments, but can be varied within the scope of the enclosed claims.

1. Method for registering a person's ability to hear, which method comprises the use of an eye tracker, wherein a person's hearing threshold is determined by emitting a sound with a determined frequency and sound intensity level to the test person, whereby the person's eye movements are followed by an eye tracker system, and a hit for said determined frequency and sound intensity level is registered when the eye tracker has registered an eye movement hit.

2. Method according to claim 1, wherein an eye movement hit is registered if the test person has fixated its gaze on a fixation image shown on a screen in front of the test person, a sound has been presented and the test person has moved its gaze to a seek area shown on a screen in front of the test person and fixated the gaze there in a sufficient time period.

3. Method according to claim 2, wherein the sufficient time period is set to 300 ms.

4. Method according to claim 1, wherein a hearing threshold is determined by stepping through sound intensity levels.

5. Method according to claim 4, wherein the stepping through sound intensity levels is performed by first giving the test person a tone of a determined intensity level and where a miss is registered, the sound intensity level is increased by 5 dB, and wherein a hit is registered, the sound intensity level is decreased by 10 dB.

6. Method according to claim 1, wherein the test starts at 40 dB, and if a miss is registered it will step up 20 dB for each miss up to 60 dB, over 60 dB the steps will be 10 dB up to 80 db and thereafter the steps will be 5 dB until a hit is registered.

7. Method according to claim 1, wherein the test starts at 40 dB and if a hit is registered, the sound intensity is lowered in steps of 20 dB until the first miss has been registered for the current frequency, and where after the sound intensity level is raised 5 dB to see if this also is registered as a hit.

8. Method according to claim 1, wherein a threshold is determined when the significance level for a sound intensity level is matching three hits in a row.

9. Method according to claim 1, wherein the sound intensity level with the lowest significance level is set as the hearing threshold.

10. Method according to claim 9, wherein the hearing threshold is set according to claim 9 if a total time limit is exceeded.

11. Method according to claim 9, wherein the total time limit is set to 900,000 ms.

12. An audiogram, wherein the audiogram consists of hearing thresholds determined according to the method of claim 1.

13. Method according to claim 9, wherein the total time limit is set to 300,000 ms.
